(19) United States
(12) Patent Application Publication
(10) Pub. No.: US 2021/0383265 A1
(43) Pub. Date: Dec. 9, 2021

(54) EMPIRICAL RISK ESTIMATION SYSTEM, EMPIRICAL RISK ESTIMATION METHOD, AND EMPIRICAL RISK ESTIMATION PROGRAM

(71) Applicant: NEC Corporation, Minato-ku, Tokyo (JP)

(72) Inventors: Daniel Gorg ANDRADE SILVA, Tokyo (JP); Yuzuru OKAJIMA, Tokyo (JP); Kunihiko SADAMASA, Tokyo (JP)

(73) Assignee: NEC Corporation, Minato-ku, Tokyo (JP)

(21) Appl. No.: 17/280,413

(22) PCT Filed: Sep. 28, 2018
(86) PCT No.: PCT/JP2018/036418

§ 371 Date: Mar. 26, 2021

(51) Int. Cl. G06N 7/00; G06N 20/00

(52) U.S. Cl. CPC G06N 7/005; G16H 40/20; G06N 20/00

(57) ABSTRACT

A density estimation unit 81 is given observed covariates and estimates a conditional probability density of a random variable, denoting the real value that is the result of a smooth function map of the unobserved covariates, by training a regression model with the response corresponding to the random variable, and the regressors corresponding to the observed covariates. An integral estimation unit 82 that estimates the one-dimensional integral of the product of a sigmoidal function with the input random variable and the conditional probability density function of the random variable.

### TECHNICAL FIELD

The present invention relates to an empirical risk estimation system, an empirical risk estimation method, and an empirical risk estimation program which estimate an expected misclassification costs of a classifier when one or more unknown covariates are acquired.

### BACKGROUND ART

In many situations, classification accuracy can be improved by collecting more covariates. However, acquiring some of the covariates might incur costs. As an example, consider the diagnosis of a patient of either having diabetes or not. Collecting information (covariates) like age and gender are almost at no cost, whereas taking blood measures clearly involve costs (e.g. working hour cost of medical doctor etc). On the other hand there is also a cost of wrongly classifying the patient as having no diabetes, although the patient is suffering from diabetes.

Therefore, it can be argued that the final goal in classification is to reduce the total cost of misclassification, which is given by the sum of acquired covariates' costs and the expected misclassification costs.

In general, it is assumed that the costs of acquiring a covariate and the costs of misclassification are given. In order to reduce the total cost of misclassification, it is necessary to estimate the expected misclassification costs when we were given more covariates (i.e. in the above example more information about the patient).

Formally, this expected cost can be expressed as

$$\begin{matrix} [ Math . 1 ] & \\ {𝔼}_{{x}_{A}} [ BayesRisk ( {x}_{A ⋃ S} ) \vert {x}_{S} ] = {𝔼}_{{x}_{A}} [ {𝔼}_{y} [ {c}_{y , δ * ( {x}_{A ⋃ S} )} \vert {x}_{A ⋃ S} ] \vert {x}_{s} ] = \int {\sum }_{y} {c}_{y , δ * ( {x}_{A ⋃ S} )} p ( y , {x}_{A} \vert {x}_{S} ) {dx}_{A} , & ( Equation 1 )\end{matrix}$$

where S denotes the set of already observed covariates, A denotes the covariates which we consider to acquire additionally. The cost of classifying a sample (i.e. in the above example the patient) as class y′, although the correct class is y, is denoted as c_{y,y′}. In the following explanation, when using a Greek letter in the text, an English notation of Greek letter may be enclosed in brackets ([ ]). In addition, when representing an upper case Greek letter, the beginning of the word in [ ] is indicated by capital letters, and when representing lower case Greek letters, the beginning of the word in [ ] is indicated by lower case letters. Moreover, note that in the following description, a Greek letter delta is described as d, and the union in mathematics is described by U in the specification. Furthermore, d*(x_{A U S}) denotes the Bayes classifier that uses the covariates A U S and is defined as

$$\begin{matrix}[ Math . 2 ] & \\ {δ}^{*} ( {x}_{A ⋃ S} ) = {argmin}_{{y}^{*} ∈ \{ 0 , 1 \}} {\sum }_{y ∈ \{ 0 , 1 \}} p ( y \vert {x}_{A ⋃ S} ) \cdot {c}_{y , {y}^{*}} , & ( Equation 2 )\end{matrix}$$

where c_{y,y*} is zero if y not equal y*, otherwise, c_{y,y}>0 specifying the cost of wrongly classifying a sample with true label y as label y*.

In the following, we call the unknown covariates A also the potential query covariates, or just simply query covariates, since these are the covariates that we might want to query (e.g. conduct clinical experiments), and then include their outcome x_{A} into the classifier.

As can be seen from Equation 1, the calculation of expected misclassification costs requires the integration over all unknown covariates A. If there are many unknown covariates, i.e. |A|>1, then the evaluation of this integral is computationally challenging, since there is no analytic closed form solution.

NPL 1 describes a Bayesian Cost-Sensitive classification method. The method described in NPL1 always limits |A| to one, and thus only a one-dimensional integral needs to be solved.

Note that NPL 2 describes a learning method using labeled data with gradient descent.

### CITATION LIST

### Non Patent Literature

[NPL 1]

Shihao Ji, Lawrence Carin, “Cost-sensitive feature acquisition and classification”, Pattern Recognition, Volume 40, Issue 5, May 2007, pp.1474-1485.

[NPL 2]

Hastie, Trevor, Tibshirani, Robert, Friedman, Jerome, “The Elements of Statistical Learning”, Springer-Verlag New York , 2009.

### SUMMARY OF INVENTION

### Technical Problem

As described above, The method described in NPL1 cannot estimate the expected misclassification costs when there are more than one query covariates. However, this can lead to the sub-optimal decision of stopping to query covariates, even though the total costs of misclassifications could be decreased further.

In the following, we given a detailed example, which shows that this is a problem even if the data is linearly separable. Let us denote by V the set of all possible covariates, S denotes the set of already observed covariates, and A denotes the covariates which we consider to acquire additionally. Let us define the total expected costs when acquiring covariates

$$[ Math . 3 ]$$

$$A ⊆ ( V ∖ S )$$

$$as$$

$$t ( A ) := {𝔼}_{{x}_{A}} [ BayesRisk ( {x}_{A ⋃ S} ) \vert {x}_{S} ] + {\sum }_{i ∈ A} {f}_{i} ,$$

where fᵢ is the cost of acquiring covariate i. The method described in NPL1 also tries to optimize t(A), but uses a greedy approach that selects the set A for which t(A) is minimal and |A|<=1. The algorithm stops, if A={0} is selected. The following example shows that a method considering only |A|<=1 can fail.

Let us consider the situation where

V\S={x₁, x₂} [Math. 4]

and the conditional joint distribution of x₁ and x₂ is an isotropic Gaussian with zero mean: p(x₁, x₂|xₛ)=N(x₁, x₂|0, I).

For simplicity, we assume the misclassification costs are c_{0,1}=c_{1,0}=c>0, and c_{y,y}=0. Furthermore, again for simplicity, the costs of querying covariate xi is assumed to be the same as x₂, which we denote by f>0.

Let us assume the following decision boundary between class 1 and class 0:

class 1⇔x₂≥mx₁+r, [Math. 5]

where without loss of generality, we assume m>0 and r>0, as illustrated in FIG. 7. FIG. 7 depicts an explanatory diagram illustrating an example of a decision boundary between classes. Furthermore, in FIG. 7, the contour plots of constant density of the conditional joint probability p(x₁, x₂|xₛ) are shown. We consider the four cases A={0}, A={x₁}, A={x₂}, and A={x₁, x₂}. For each A we calculate the expected misclassification costs, which we denote as [alpha]_{A}.

First, let A={x₁, x₂}, then

$$\begin{matrix} [ Math . 6 ] \\ {δ}^{*} ( {x}_{A ⋃ S} ) = \{ \begin{matrix}1 & if {x}_{2} \geq {mx}_{1} + r , \\ 0 & else .\end{matrix} \\ and \\ [ Math . 7 ] \\ {a}_{( {x}_{1} , {x}_{2} )} := {𝔼}_{{x}_{A}} [ BayesRisk ( {x}_{A ⋃ S} ) \vert {x}_{S} ] = \int ( {\sum }_{y} {c}_{y , δ * ( {x}_{A ⋃ S} )} p ( y \vert {x}_{A} , {x}_{S} ) ) p ( {x}_{A} \vert {x}_{S} ) {1}_{{x}_{2} \geq {mx}_{1} + r} ( {x}_{A} ) {dx}_{A} + \int ( {\sum }_{y} {c}_{y , δ * ( {x}_{A ⋃ S} )} p ( y \vert {x}_{A} , {x}_{S} ) ) p ( {x}_{A} \vert {x}_{S} ) {1}_{{x}_{2} \geq {mx}_{1} + r} ( {x}_{A} ) {dx}_{A} = \int ( {\sum }_{y} {c}_{y , 1} p ( y \vert {x}_{A} , {x}_{S} ) ) p ( {x}_{A} \vert {x}_{S} ) {1}_{{x}_{2} \geq {mx}_{1} + r} ( {x}_{A} ) {dx}_{A} + \int ( {\sum }_{y} {c}_{y , 0} p ( y \vert {x}_{A} , {x}_{S} ) ) p ( {x}_{A} \vert {x}_{S} ) {1}_{{x}_{2} \geq {mx}_{1} + r} ( {x}_{A} ) {dx}_{A} = \int ( {c}_{0 , 1} p ( y = 0 \vert {x}_{A} , {x}_{S} ) ) p ( {x}_{A} \vert {x}_{S} ) {1}_{{x}_{2} \geq {mx}_{1} + r} ( {x}_{A} ) {dx}_{A} + \int ( {c}_{1 , 0} p ( y = 1 \vert {x}_{A} , {x}_{S} ) ) p ( {x}_{A} \vert {x}_{S} ) {1}_{{x}_{2} \geq {mx}_{1} + r} ( {x}_{A} ) {dx}_{A} = 0.\end{matrix}$$

Next, let A={x₁}, then we have

$$[ Math . 8 ]$$

$$p ( y = 1 \vert {x}_{1} , {x}_{S} ) = p ( {x}_{2} \geq {mx}_{1} + r \vert {x}_{1} , {x}_{S} ) = {\int }_{{mx}_{1} + r}^{∞} N ( {x}_{2} \vert 0 , 1 ) {dx}_{2} . \\ {δ}^{*} ( {x}_{A ⋃ S} ) = \{ \begin{matrix}1 & if {\int }_{{mx}_{1} + r}^{∞} N ( {x}_{2} \vert 0 , 1 ) {dx}_{2} \geq 0.5 , \\ 0 & else .\end{matrix}$$

Define b as the value of x₁ for which

∫ₘₓ_{₁}₊ᵣ^{∞}N(x₂|0, 1)dx₂=0.5. [Math. 9]

Since

∫₀^{∞}N(x₂|0, 1)dx₂=0.5 [Math. 10]

we have b=−r/m. we have

$$[ Math . 11 ]$$

$${α}_{( {x}_{1} )} := {𝔼}_{{x}_{A}} [ BayesRisk ( {x}_{A ⋃ S} ) \vert {x}_{S} ] = \int ( {\sum }_{y} {c}_{y , δ * ( {x}_{1} , {x}_{S} )} p ( y \vert {x}_{1} , {x}_{S} ) ) p ( {x}_{1} \vert {x}_{S} ) {dx}_{1} = {\int }_{- ∞}^{b} ( {\sum }_{y} {c}_{y , 1} p ( y \vert {x}_{1} , {x}_{S} ) ) p ( {x}_{1} \vert {x}_{S} ) {dx}_{1} + {\int }_{b}^{∞} ( {\sum }_{y} {c}_{y , 0} p ( y \vert {x}_{1} , {x}_{S} ) ) p ( {x}_{1} \vert {x}_{S} ) d {x}_{1} = {\int }_{- ∞}^{b} ( {c}_{0 , 1} p ( y = 0 \vert {x}_{1} , {x}_{S} ) ) p ( {x}_{1} \vert {x}_{S} ) {dx}_{1} + {\int }_{b}^{∞} ( {c}_{1 , 0} p ( y = 1 \vert {x}_{1} , {x}_{S} ) ) p ( {x}_{1} \vert {x}_{S} ) {dx}_{1} .$$

Analogously, we can calculate the expected Bayes Risk {x₂}.

Finally, let A={0}. Let us define the random variable z :=x₂−mx₁−r. Since x₁ and x₂ are independent standard normal variavles, we have z˜N(−r, m²+1). We therefore have

$$[ Math . 12 ]$$

$$p ( y = 1 \vert {x}_{S} ) = p ( {x}_{2} \geq {mx}_{1} + r \vert {x}_{S} ) = p ( {x}_{2} - m {x}_{1} - r \geq 0 \vert {x}_{S} ) = p ( z \geq 0 ) = {\int }_{0}^{∞} N ( z \vert - r , {m}^{2} + 1 ) dz < 0.5 ,$$

since we assume r>0. Therefore, we have d*(xₛ)=0. And, as a consequence, we have

$$\begin{matrix}{α}_{\{ θ \}} \text{:} = {𝔼}_{{x}_{A}} [ Bayes Risk ( {x}_{AUS} ) \vert {x}_{S} ] = {\sum }_{y} {c}_{y} , {δ}^{*} ( {x}_{S} ) p ( y \vert {x}_{S} ) \\ = {\sum }_{y} {c}_{y , 0} p ( y \vert {x}_{S} ) \\ = {c}_{1 , 0} p ( y = 1 \vert {x}_{S} ) .\end{matrix} \qquad [ Math . 13 ]$$

Without loss of generality, let us assume that [alpha]_{{x1}}<[alpha]_{{x2}}, and the costs of each covariate is f>0. The greedy algorithm with |A|<=1, fails if (I) t({0})<t({x₁}) or (II) t({0})>t({x₁, x₂}). That means that (I) [alpha]_{{0}}<[alpha]_{{x1}}f or (II) [alpha]_{{0}}>2f, which is equivalent to [alpha]_{{x1}}>[alpha]_{{0}}/2.

Therefore, except for the case r=0, there is always a covariate cost f>0 for which the greedy algorithm will fail. As a concrete numerical example, let us assume that r=m=1, c_{0,1}=c_{1,0}=100, and f=10. The total expected costs for each query set are listed in Table 1.

TABLE 1

| A | t(A) |
|---|---|
| {∅} | 24.0 |
| {x₂} | 28.2 |
| {x₂} | 28.2 |
| {x₁, x₂} | 20.0 |

It is an exemplary object of the present invention to provide an empirical risk estimation system, an empirical risk estimation method, and an empirical risk estimation program which, even when the number of query covariates is more than one, can estimate an empirical risk with high accuracy at low computational costs.

### Solution to Problem

A empirical risk estimation system according to the present invention includes: a density estimation unit that estimates a conditional probability density of a random variable, denoting the real value that is the result of a smooth function map of given unobserved covariates, by training a regression model with the response corresponding to the random variable, and the regressors corresponding to the observed covariates; and an integral estimation unit that estimates the one-dimensional integral of the product of a sigmoidal function with the input random variable and the conditional probability density function of the random variable.

A empirical risk estimation method according to the present invention includes: estimating a conditional probability density of a random variable, denoting the real value that is the result of a smooth function map of given unobserved covariates, by training a regression model with the response corresponding to the random variable, and the regressors corresponding to the observed covariates; and estimating the one-dimensional integral of the product of a sigmoidal function with the input random variable and the conditional probability density function of the random variable.

A empirical risk estimation program according to the present invention causes a computer to perform: a density estimation process of estimating a conditional probability density of a random variable, denoting the real value that is the result of a smooth function map of given unobserved covariates, by training a regression model with the response corresponding to the random variable, and the regressors corresponding to the observed covariates; and an integral estimation process of estimating the one-dimensional integral of the product of a sigmoidal function with the input random variable and the conditional probability density function of the random variable.

### Advantageous Effects of Invention

According to the present invention, even when the number of query covariates is more than one, it is possible to estimate an empirical risk with high accuracy at low computational costs.

### BRIEF DESCRIPTION OF DRAWINGS

FIG. 1 It depicts an exemplary block diagram illustrating the structure of an exemplary embodiment of an empirical risk estimation system according to the present invention.

FIG. 2 It depicts an exemplary explanatory diagram illustrating the structure of an exemplary embodiment of the empirical risk estimation system according to the present invention.

FIG. 3 It depicts an exemplary explanatory diagram illustrating different Sigmoid function approximations.

FIG. 4 It depicts a flowchart illustrating an operation example of the empirical risk estimation system in this exemplary embodiment.

FIG. 5 It depicts a block diagram illustrating an outline of an empirical risk estimation system according to the present invention.

FIG. 6 It depicts a schematic block diagram illustrating the configuration example of the computer according to the exemplary embodiment of the present invention.

FIG. 7 It depicts depicts an explanatory diagram illustrating an example of a decision boundary between classes.

### DESCRIPTION OF EMBODIMENTS

The following describes an exemplary embodiment of the present invention with reference to drawings.

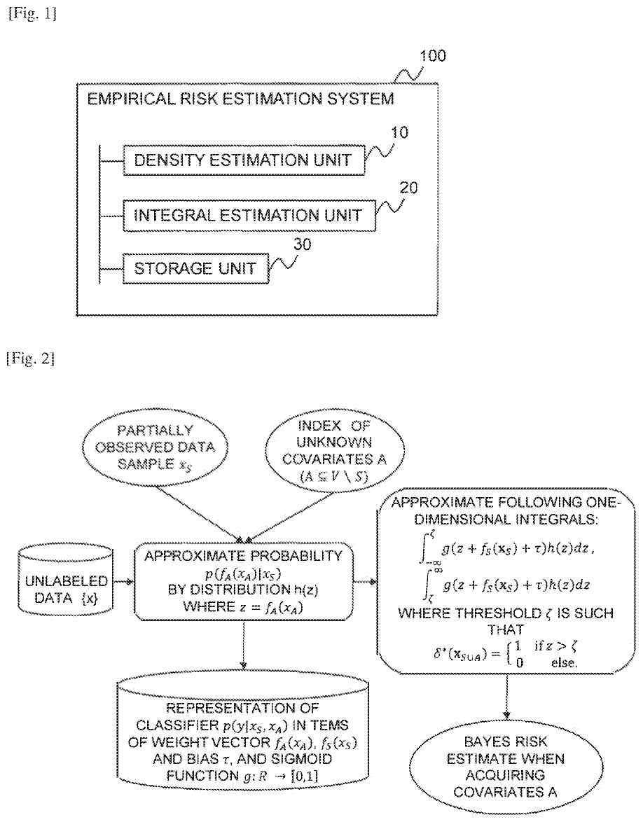

FIG. 1 is an exemplary block diagram illustrating the structure of an exemplary embodiment of an empirical risk estimation system according to the present invention. FIG. 2 is an exemplary explanatory diagram illustrating the structure of an exemplary embodiment of the empirical risk estimation system according to the present invention.

In the exemplary present embodiment, it is assumed that the conditional class probability can be expressed as the following generalized additive model.

p(y|x_{A}, X_{S})=g(f_{A}(x_{A})+f_{S}(x_{S})+τ), [Math. 14]

where g is a sigmoid function, e.g. the logistic function, a greek letter tau (hereinafter [tau]) is the bias, and f_{A}:R^{|A|}->R, and, f_{S}:R^{|S|->R are arbitrary smooth functions. The method of learning [tau] and the functions is arbitrary, and for example, commonly, the [tau] and the functions have been learned using labeled data with gradient descent. The method described in NPL} 2 may be used for learning. In the exemplary present embodiment, it is assumed that the [tau] and the functions to be given.

For example in the case of a classifier with linear decision boundary, we have

p(y|x_{A}, x_{S})=g(β^{T}x+τ)=g(β_{A}^{T}x_{A}+β_{S}^{T}x_{S}+τ) [Math. 15]

where a greek letter beta (hereinafter [beta]) is the weight vector of the classifier that was learned from labeled data. We remark that [beta]_{A} and [beta]_{S} denote the sub-vectors of [beta] corresponding to the covariates A and S, respectively.

The expected misclassification costs can be expressed as follows.

$$\begin{matrix}[ Math . 16 ] & \\ \begin{matrix}\begin{matrix}{𝔼}_{{x}_{A}} [ Bayes Risk ( {x}_{AUS} ) \vert {x}_{S} ] = \int {\sum }_{y} {c}_{y} , {δ}^{*} ( {x}_{AUS} ) p ( y , {x}_{A} \vert {x}_{S} ) {dx}_{A} \\ = \int {\sum }_{y} {c}_{y} , {δ}^{*} ( {x}_{AUS} ) p ( y \vert {x}_{A} , {x}_{S} ) p ( {x}_{A} \vert {x}_{S} ) {dx}_{A} \\ = \int {\sum }_{y} {c}_{y} , {δ}^{*} ( {f}_{A} ( {x}_{A} ) , {f}_{S} ( {x}_{S} ) ) p ( y \vert {f}_{A} ( {x}_{A} ) , \\ {f}_{S} ( {x}_{S} ) ) p ( {x}_{A} \vert {x}_{S} ) {dx}_{A} \\ = \int {\sum }_{y} {c}_{y} , {δ}^{*} ( z , {f}_{S} ( {x}_{S} ) ) p ( y \vert z , {f}_{S} ( {x}_{S} ) ) h ( z ) dz ,\end{matrix} & \end{matrix} & ( Equation 3 )\end{matrix}$$

where we introduced the random variable z :=f_{A}(x_{A}), with density h(z) :=p(z|x_{S}). The resulting integral in Equation 3 is only a one-dimensional integral in z. However, it requires us to estimate h(z).

The empirical risk estimation system 100 according to the present exemplary embodiment includes a density estimation unit 10, an integral estimation unit 20 and a storage unit 30.

The density estimation unit 10 estimates the h(z). In particular, the density estimation unit 10 is given the observed covariates S, and estimates a conditional probability density of a random variable z by training a regression model with the response corresponding to z, and the regressors corresponding to the covariates S. The z is denoting the real value that is the result of a smooth function map of the unobserved covariates A.

In the following, it is explained how h(z) can be estimated using linear or non-linear regression. Let us denote by {x⁽ⁱ⁾}ⁿᵢ₌₁ the collection of unlabeled data. Note that the density estimation unit 10 does not require class-labeled data. From the collection of unlabeled data, the density estimation unit 10 may form a collection of response and explanatory variable pairs of the form {(z⁽ⁱ⁾, x_{S}⁽ⁱ⁾)}ⁿᵢ₌₁, where z⁽ⁱ⁾=f_{A}(x_{A}⁽ⁱ⁾). For example, if it is assumed that a linear relationship between z and x_{S} with normal noise, then the density estimation unit 10 has

p(z|x_{S})=N(γ^{T}x_{S}, σ²), [Math. 17]

for some parameter vector

γ ∈ [symbol] ^{|S|} and σ² ∈ [symbol] [Math. 18]

which can be estimated from the data {(z⁽ⁱ⁾, x_{S}⁽ⁱ⁾)}ⁿᵢ₌₁. Note that a greek letter mu is denoted as [mu], an uppercase greek letter Sigma is denoted as [Sigma], and a lowercase greek letter sigma is denoted as [sigma]. For example, if the joint distribution p(x) is a multivariate normal distribution N([mu], [Sigma]), and p(y|x_{A}, x_{B}) follows a logistic regression model with weight vector b, then the maximum likelihood estimate leads to

p(z|x_{S})=N(β_{A}^{T}μ_{A|S}, β_{A}^{T}Σ_{A|S}β_{A}), [Math. 19]

with

μ_{A|S}=μ_{A}+Σ_{A,S}Σ_{S,S}⁻¹(x_{S}−μ_{S}),

Σ_{A|S}=Σ_{A,A}−Σ_{A,S}Σ_{S,S}⁻¹Σ_{S,A}.

That is, the density estimation unit 10 may estimate the conditional probability density of z by the normal distribution.

If a linear relationship between z and x_{S} is unreasonable, then a non-parametric regression model like Gaussian processes might be more appropriate. As before, let x⁽ⁱ⁾ (x⁽ⁱ⁾ belongs to R^{p}) be the i-th sample of x available at training time, and let x*_{S} be the observed covariates of a new sample at test time. Then the matrix K(X_{S}, X_{S}) is defined as follows

K(X_{S}, X_{S})ᵢⱼ=k(x_{S}⁽ⁱ⁾, x_{S}^{(j)}), [Math. 20]

where k is a covariance function, for example, using the exponential squared covariance function the density estimation unit 10 has

$$k ( {x}_{S}^{( i )} , {x}_{S}^{( j )} ) = {e}^{- \frac{{‖ {x}_{S}^{( i )} - {x}_{S}^{( j )} ‖}_{2}^{2}}{{l}^{2}}} , \qquad [ Math . 21 ]$$

where 1 is the length scale parameter. Furthermore, the density estimation unit 10 defines the column vector z (z belongs to Rⁿ) as

zᵢ=f_{A}(x_{A}⁽ⁱ⁾). [Math. 22]

And for a new sample x*, at test time, the density estimation unit 10 defines, analogously

z*=f_{A}(x*_{A}) [Math. 23]

Finally, the density estimation unit 10 defines the column vector k(x*_{S}, X_{S}) (k(x*_{S}, X_{S}) belongs to Rⁿ) as follows

k(x*_{S}, X_{S})ᵢ=k(x*_{S}, x_{S}⁽ⁱ⁾). [Math. 24]

Then under the Gaussian process assumption with additional Gaussian noise with variance [sigma]₀², the density estimation unit 10 has

$$( \begin{matrix}z \\ {z}^{*}\end{matrix} ) = N ( ( \begin{matrix}{μ}_{0} {1}_{n} \\ {μ}_{0}\end{matrix} ) , ( \begin{matrix}K ( {X}_{S} , {X}_{S} ) + {σ}_{0}^{2} {I}_{n} & k ( {x}_{S}^{*} , {X}_{S} ) \\ {k ( {x}_{S}^{*} , {X}_{S} )}^{T} & k ( {x}_{S}^{*} , {x}_{S}^{*} )\end{matrix} ) ) , \qquad [ Math . 25 ]$$

where the density estimation unit 10 assumes a fixed mean [mu]₀ given by

$${μ}_{0} = \frac{1}{n} {{\sum }^{n}}_{i = 1} {z}_{i} , \qquad [ Math . 26 ]$$

and 1ₙ (1ₙ belongs to Rⁿ) is the vector with all one. As a consequence the density estimation unit 10 has

h(z)=N(μ, σ²), [Math. 27]

with

μ=μ₀+k(x*_{S}, X_{S})^{T}(K(X_{S}, X_{S})+σ₀²Iᵤ)⁻¹(t−1μ₀),

σ²=k(x*_{S}, x*_{S})−k(x*_{S}, X_{S})^{T}(K(X_{S}, X_{S})+σ₀²Iᵤ)⁻¹k(x*_{S}, X_{S}).

The integral estimation unit 20 estimates Equation 3. In particular, the integral estimation unit 20 estimates the one-dimensional integral of the product of a sigmoidal function g with input z and the conditional probability density function of z.

The integral estimation unit 20 may simply use Monte Carlo samples from h(z) in order to estimate Equation 3. On the other hand, in order to improve the processing speed, the integral estimation unit 20 may use a different strategy based on a piece-wise linear approximation of the sigmoid function g as explained in the following.

First, the integral estimation unit 20 expresses the expected misclassification cost as follows

$$\begin{matrix}{𝔼}_{{x}_{A}} [ Bayes Risk ( {x}_{AUS} ) \vert {x}_{S} ] = {𝔼}_{{x}_{A}} [ {\sum }_{y} {c}_{y} , {δ}^{*} ( {x}_{AUS} ) p ( y \vert {x}_{AUS} ) \vert {x}_{S} ] \\ = {𝔼}_{{x}_{A}} [ {c}_{0} , {δ}^{*} ( {x}_{AUS} ) p ( y = 0 \vert {x}_{AUS} ) + \\ {c}_{1} , {δ}^{*} ( {x}_{AUS} ) p ( y = 1 \vert {x}_{AUS} ) \vert {x}_{S} ] \\ = {𝔼}_{{x}_{A}} [ {c}_{0} , {δ}^{*} ( {x}_{AUS} ) p ( y = 0 \vert {x}_{AUS} ) \vert {x}_{S} ] + \\ {𝔼}_{{x}_{A}} [ {c}_{1} , {δ}^{*} ( {x}_{AUS} ) p ( y = 1 \vert {x}_{AUS} ) \vert {x}_{S} ] .\end{matrix} \qquad [ Math . 28 ]$$

Next, note that

δ*(x_{A∪S})=argmin[p(y=1|x_{A∪S})·c_{1,0}, p(y=0|x_{A∪S})·c_{0,1}]. [Math. 29]

Furthermore, the integral estimation unit 20 has

$$\begin{matrix}{δ}^{*} ( {x}_{AUS} ) = 1 ⇔ \frac{p ( y = 1 \vert {x}_{AUS} ) \cdot {c}_{1 , 0}}{p ( y = 1 \vert {x}_{AUS} ) \cdot {c}_{0 , 1}} \geq 1 \\ ⇔ \frac{g ( {f}_{A} ( {x}_{A} ) + {f}_{S} ( {x}_{S} ) + τ ) \cdot {c}_{1.0}}{( 1 - g ( {f}_{A} ( {x}_{A} ) + {f}_{S} ( {x}_{S} ) + τ ) ) \cdot {c}_{0 , 1}} \geq 1 \\ ⇔ {e}^{{f}_{A} ( {x}_{A} ) + {f}_{S} ( {x}_{S} ) + τ} \geq \frac{{c}_{0.1}}{{c}_{1 , 0}} \\ ⇔ {f}_{A} ( {x}_{A} ) \geq log ( \frac{{c}_{0 , 1}}{{c}_{1 , 0}} ) - τ - {f}_{S} ( {x}_{S} ) \\ ⇔ z \geq ζ ,\end{matrix} \\ where \\ z \text{:} = {f}_{A} ( {x}_{A} ) , and ζ \text{:} = log ( \frac{{c}_{0 , 1}}{{c}_{1 , 0}} ) - τ - {f}_{S} ( {x}_{S} ) \qquad [ Math . 30 ]$$

As described above, d*(x_{A U S}) depends only z (random variable) and [zeta] (fixed). Thus, the integral estimation unit 20 has

$$\begin{matrix}{𝔼}_{{x}_{A}} [ {c}_{1 ,} {δ}^{*} ( {x}_{AUS} ) p ( y = 1 \vert {x}_{AUS} ) \vert {x}_{S} ] = \int {c}_{1 , {δ}^{*} ( x , ζ )} g ( z + {f}_{S} ( {x}_{S} ) + τ ) h ( z ) dz \\ = {\int }_{- ∞}^{ζ} {c}_{1 , 0} g ( z + {f}_{S} ( {x}_{S} ) + τ ) h ( z ) dz + \\ {\int }_{ζ}^{∞} {c}_{1 , 1} g ( z + {f}_{S} ( {x}_{S} ) + τ ) h ( z ) dz \\ = {c}_{1 , 0} {\int }_{- ∞}^{ζ} g ( z + {f}_{S} ( {x}_{S} ) + τ ) h ( z ) dz + \\ {c}_{1 , 1} {\int }_{ζ}^{∞} g ( z + {f}_{S} ( {x}_{S} ) + τ ) h ( z ) dz \\ = {c}_{1 , 0} {\int }_{- ∞}^{ζ} g ( z + {f}_{S} ( {x}_{S} ) + τ ) h ( z ) dz .\end{matrix} \qquad [ Math . 31 ]$$

And, analogously, the integral estimation unit 20 has

$$\begin{matrix}{𝔼}_{{x}_{A}} [ {c}_{0 ,} {δ}^{*} ( {x}_{AUS} ) p ( y = 0 \vert {x}_{AUS} ) \vert {x}_{S} ] = {c}_{0 , 0} {\int }_{- ∞}^{ζ} ( 1 - g ( z + {f}_{S} ( {x}_{S} ) + τ ) ) h ( z ) dz + \\ {c}_{0 , 1} {\int }_{ζ}^{∞} ( 1 - g ( z + {f}_{S} ( {x}_{S} ) + τ ) ) h ( z ) dz \\ = {c}_{0 , 1} {\int }_{ζ}^{∞} ( 1 - g ( z + {f}_{S} ( {x}_{S} ) + τ ) ) h ( z ) dz \\ = {c}_{0 , 1} {\int }_{ζ}^{∞} h ( z ) dz - {c}_{0 , 1} {\int }_{ζ}^{∞} g ( z + {f}_{S} ( {x}_{S} ) + τ ) h ( z ) dz .\end{matrix} \qquad [ Math . 32 ]$$

Thus the remaining task is to evaluate the following integral

[Math. 33]

∫_{a′}^{b′}g(z+f_{S}(x_{S})+τ)h(z)dz=∫_{a′+f}_{_{S}}₍ₓ_{_{S}}_{)+τ}^{b′+f}^{_{S}}^{(x}^{_{S}}^{)+τ}g(u)h(u−f_{S}(x_{S})−τ)du. (Equation 4)

One popular strategy is to approximate the sigmoid function g by the cumulative distribution function of the standard normal distribution [Phi]. However, it turns out that this approximation does not work here, since a or b is bounded in our case. Instead, in the exemplary present embodiment, the integral estimation unit 20 uses here the fact that the sigmoid function can be well approximated with only a few number of linear functions. It is assumed that h(z) is a normal distribution with mean [mu]′ and variance [sigma]². In order to facilitate notation, the following constants are introduced.

a :=a′+f_{S}(x_{S})+τ,

b :=b′+f_{S}(x_{S})+τ,

μ :=μ+f_{S}(x_{S})+τ. [Math. 34]

Then the integral in Equation 4 can be written as

$$\begin{matrix}[ Math . 35 ] & \\ {\int }_{a}^{b} g ( u ) \frac{1}{\sqrt{2 π {σ}^{2}}} {e}^{- \frac{1}{2 {σ}^{2}} {( u - μ )}^{2}} du . & ( Equation 5 )\end{matrix}$$

The integral estimation unit 20 has define the following piece-wise linear approximation of the sigmoid function

$$g ( u ) \approx {\sum }_{t = 1}^{ξ + 2} ( {1}_{[ {b}_{t - 1} , {b}_{t} ]} ( u ) ( {m}_{t} u + {v}_{t} ) ) , \\ where \\ for 1 \leq t \leq ξ + 1 \\ {b}_{t} \text{:} = - 10 + \frac{20}{ξ} ( t - 1 ) \\ and \\ for 1 \leq t \leq ξ \\ {m}_{t + 1} \text{:} = \frac{g ( {b}_{t + 1} ) - g ( {b}_{t} )}{{b}_{t + 1} - {b}_{t}} , \\ {v}_{t + 1} \text{:} = g ( {b}_{t} ) - {m}_{t + 1} {b}_{t} , \\ and \\ {b}_{0} \text{:} = - ∞ , {m}_{1} \text{:} = 0 , {v}_{1} \text{:} = g ( {b}_{1} ) , \\ {b}_{ξ + 2} \text{:} = + ∞ , {m}_{ξ + 2} \text{:} = 0 , {v}_{ξ + 2} \text{:} = g ( {b}_{ξ + 1} ) , \qquad [ Math . 36 ]$$

and [xi] is the number of linear approximations, which is, for example, set to 40. A comparison with the approximation

$$Φ ( \sqrt{\frac{π}{8}} u ) \qquad [ Math . 37 ]$$

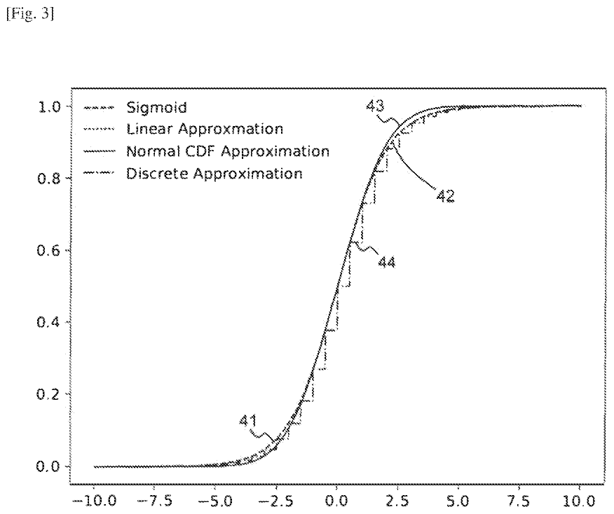

is shown in FIG. 3. FIG. 3 depicts an exemplary explanatory diagram illustrating different Sigmoid function approximations. In FIG. 3, a line 41 represents a Sigmoid, a line 42 represents a linear approximation, a line 43 represents a normal CDF (cumulative distribution function) approximation, and a line 44 represents a discrete approximation. According to NPL1, for the linear function approximation and the discrete bin approximation, [xi]=40 is set. For the normal CDF approximation,

$$Φ ( \sqrt{\frac{π}{8}} u ) \qquad [ Math . 38 ]$$

is used.

This shows that for a relative few number of linear approximations, the integral estimation unit 20 can achieve an approximation that is more accurate than the [Phi]-approximation. More importantly, as shown below, this allows for a tractable calculation of the integral in Equation 5, which is not the case when using the [Phi]-approximation.

Then the integral estimation unit 20 has

$$\begin{matrix}{\int }_{a}^{b} g ( u ) \frac{1}{\sqrt{2 π {σ}^{2}}} {e}^{- \frac{1}{2 {σ}^{2}} {( u - μ )}^{2}} du = {\int }_{a}^{b} {\sum }_{t = 1}^{ξ + 2} ( {1}_{[ {b}_{t - 1} , {b}_{t} ]} ( u ) ( {m}_{t} u + {v}_{t} ) ) \frac{1}{\sqrt{2 π {σ}^{2}}} {e}^{- \frac{1}{2 {σ}^{2}} {( u - μ )}^{2}} du \\ = {\sum }_{t = 1}^{ξ + 2} {\int }_{max ( a , {b}_{t - 1} )}^{min ( b , {b}_{t} )} ( {m}_{t} u + {v}_{t} ) \frac{1}{\sqrt{2 π {σ}^{2}}} {e}^{- \frac{1}{2 {σ}^{2}} {( u - μ )}^{2}} du \\ = {\sum }_{t = 1}^{ξ + 2} {m}_{t} {\int }_{max ( a , {b}_{t - 1} )}^{min ( b , {b}_{t} )} u \frac{1}{\sqrt{2 π {σ}^{2}}} {e}^{- \frac{1}{2 {σ}^{2}} {( u - μ )}^{2}} du + \\ {v}_{t} {Φ}_{max ( a , {b}_{t - 1} )}^{min ( b , {b}_{t} )} ,\end{matrix} \\ where \\ {Φ}_{l}^{o} \text{:} = {\int }_{l}^{o} \frac{1}{\sqrt{2 π {σ}^{2}}} {e}^{- \frac{1}{2 {σ}^{2}} {( u - μ )}^{2}} du \qquad [ Math . 39 ]$$

which can be well approximated with standard implementations. The remaining integral can also be expressed by [Phi] using the substitution u−[mu] :=r, the integral estimation unit 20 has

$$\begin{matrix}{\int }_{l}^{∘} u \frac{1}{\sqrt{2 π {σ}^{2}}} {e}^{- \frac{1}{2 {σ}^{2}} {( u - μ )}^{2}} du = {\int }_{l - μ}^{o - μ} ( r + μ ) \frac{1}{\sqrt{2 {πσ}^{2}}} {e}^{- \frac{1}{2 {σ}^{2}} {r}^{2}} dr \\ = {\int }_{l - μ}^{o - μ} r \frac{1}{\sqrt{2 {πσ}^{2}}} {e}^{- \frac{1}{2 {σ}^{2}} {r}^{2}} dr + {μΦ}_{l}^{o} \\ = {\frac{1}{\sqrt{2 {πσ}^{2}}} [ - {σ}^{2} {e}^{- \frac{1}{2 {σ}^{2}} {r}^{2}} ]}_{l - μ}^{o - μ} + {μΦ}_{l}^{o} \\ = \frac{σ}{\sqrt{2 π}} ( {e}^{- \frac{1}{2 {σ}^{2}} {( l - μ )}^{2}} - {e}^{- \frac{1}{2 {σ}^{2}} {( o - μ )}^{2}} ) + {μΦ}_{l}^{o} .\end{matrix} \qquad [ Math . 40 ]$$

In this way, the integral estimation unit 20 may estimate the one-dimensional integral by using a piece-wise linear approximation of the sigmoid function.

The storage unit 30 stores various data. The storage unit 30 may store unlabeled Data {x}. The storage unit 30 is realized by a magnetic disk or the like.

The density estimation unit 10 and the integral estimation unit 20 are each implemented by a CPU of a computer that operates in accordance with a program (empirical risk estimation program). For example, the program may be stored in a storage unit 30 included in the empirical risk estimation system 100, and the CPU may read the program and operate as the density estimation unit 10 and the integral estimation unit 20 in accordance with the program.

In the empirical risk estimation system of the exemplary present embodiment, the density estimation unit 10 and the integral estimation unit 20 may each be implemented by dedicated hardware. Further, the empirical risk estimation system according to the present invention may be configured with two or more physically separate devices which are connected in a wired or wireless manner.

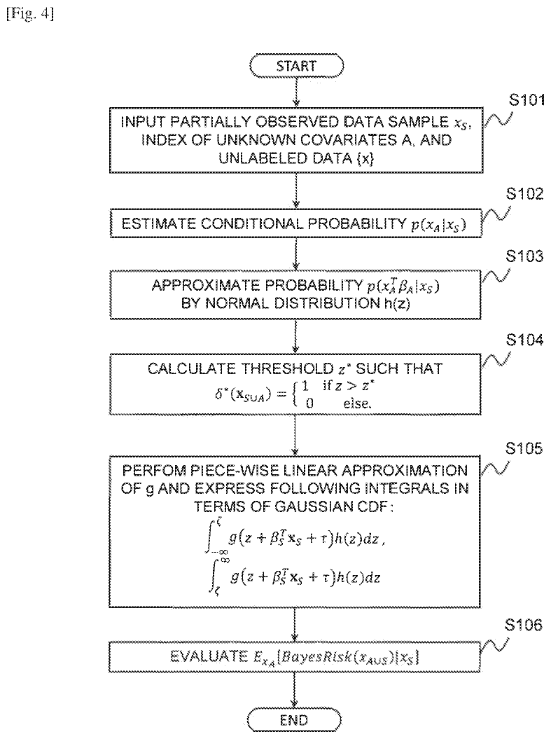

The following describes an example of the empirical risk estimation system in this exemplary embodiment. FIG. 4 is a flowchart illustrating an operation example of the empirical risk estimation system in this exemplary embodiment.

The density estimation unit 10 inputs partially observed data sample x_{S}, index of unknown covariates A, and unlabeled Data {x} (Step S101). The density estimation unit 10 estimates conditional probability p(x_{A}|x_{S}) (Step S102). The density estimation unit 10 approximates probability p(x^{T}_{A}[beta]_{A}|x_{S}) by a normal distribution h(z) (Step S103).

The integral estimation unit 20 calculate threshold z* such that if z>z* then d*(x_{S U A})=1 else d*(x_{S U A})=0. (Step S104). The integral estimation unit 20 performs piece-wise linear approximation of g and express the following integrals in terms of Gaussian CDF (Step S105):

∫_{z*}^{∞}g(z+β_{S}^{T}x_{S}+τ))h(z)dz,

∫_{-∞}^{z*}g(z+β_{S}^{T}x_{S}+τ))h(z)dz. [Math. 41]

The integral estimation unit 20 evaluates E_{xA}[BayesRisk(x_{A U S})|x_{S}] (Step S106). In this way, the covariates A is acquired, Bayes Risk is estimated.

In this manner, in the present exemplary embodiment, the density estimation unit 10 estimates a conditional probability density of z by training a regression model with the response corresponding to z, and the regressors corresponding to the observed covariates S. Then the integral estimation unit 20 estimates the one-dimensional integral of the product of a sigmoidal function g with input z and the conditional probability density function of z.

With the above structure, even when the number of query covariates is more than one, it is possible to estimate an empirical risk with high accuracy at low computational costs.

That is, in the exemplary present embodiment, a classifier which class probability is a additive function of the feature map of the query covariates is considered, and the value of the sum of those feature maps is a real-valued number. This real-valued number is considered as a random variable for which we directly estimate the conditional distribution given the already observed covariates. Then the integral estimation unit 20 estimates the expected misclassification costs with respect to this conditional distribution.

In this case, in the exemplary present embodiment, even when the number of query covariates is more than one, it is only necessary to solve a one-dimensional integral in order to estimate the expected misclassification costs. Therefore, in contrast to high dimensional integrals, the one dimensional integral can be solved with numeric methods with high accuracy at low computational costs.

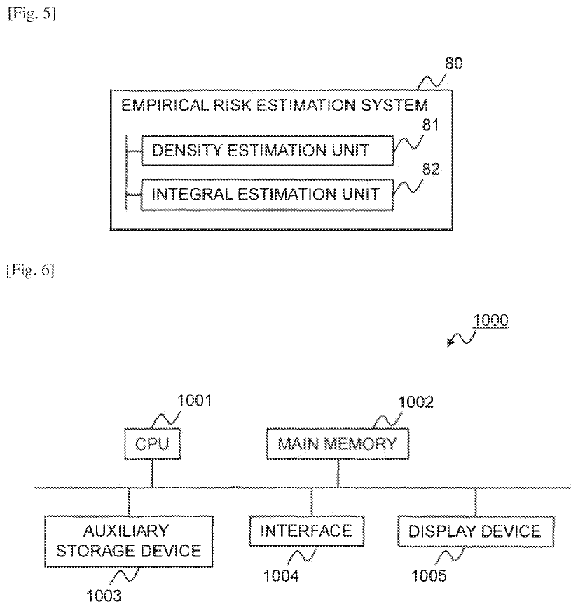

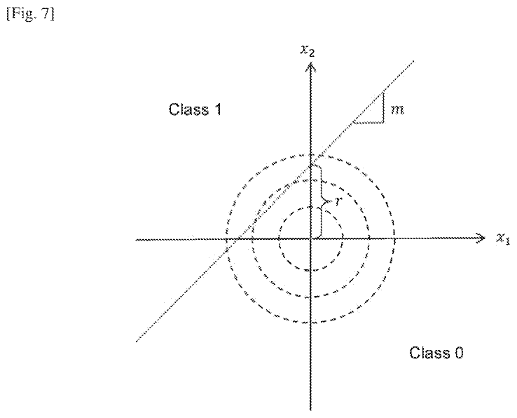

Next, an outline of the present invention will be described. FIG. 5 is a block diagram illustrating an outline of the empirical risk estimation system according to the present invention. The empirical risk estimation system 80 (for example, empirical risk estimation system 100) according to the present invention includes: a density estimation unit 81 (for example, the density estimation unit 10) that is given observed covariates (for example, S) and estimates a conditional probability density of a random variable (for example z), denoting the real value that is the result of a smooth function map of the unobserved covariates (for example A), by training a regression model with the response corresponding to the random variable (for example z), and the regressors corresponding to the observed covariates (for example, S); and an integral estimation unit 82 (for example, integral estimation unit 20) that estimates the one-dimensional integral of the product of a sigmoidal function (for example g) with the input random variable (for example z) and the conditional probability density function of the random variable (for example z).

With such a configuration, even when the number of query covariates is more than one, it is possible to estimate an empirical risk with high accuracy at low computational costs.

In addition, the density estimation unit 81 may estimate the conditional probability density of the random variable (for example z) by a normal distribution, and the integral estimation unit 82 may estimate the one-dimensional integral by using a piece-wise linear approximation of the sigmoid function. With such a configuration, it is possible to improve the processing speed.

Next, a configuration example of a computer according to the exemplary embodiment of the present invention will be described. FIG. 6 is a schematic block diagram illustrating the configuration example of the computer according to the exemplary embodiment of the present invention. The computer 1000 includes a CPU 1001, a main memory 1002, an auxiliary storage device 1003, an interface 1004, and a display device 1005.

The empirical risk estimation system 100 described above may be installed on the computer 1000. In such a configuration, the operation of the system may be stored in the auxiliary storage device 1003 in the form of a program. The CPU 1001 reads a program from the auxiliary storage device 1003 and loads the program into the main memory 1002, and performs a predetermined process in the exemplary embodiment according to the program.

The auxiliary storage device 1003 is an example of a non-transitory tangible medium. Another example of the non-transitory tangible medium includes a magnetic disk, a magnetooptical disk, a CD-ROM, a DVD-ROM, a semiconductor memory or the like connected through the interface 1004. Furthermore, when this program is distributed to the computer 1000 through a communication line, the computer 1000 receiving the distributed program may load the program into the main memory 1002 to perform the predetermined process in the exemplary embodiment.

Furthermore, the program may partially achieve the predetermined process in the exemplary embodiment. Furthermore, the program may be a difference program combined with another program already stored in the auxiliary storage device 1003 to achieve the predetermined process in the exemplary embodiment.

Furthermore, depending on the content of a process according to an exemplary embodiment, some of elements of the computer 1000 can be omitted. For example, when information is not presented to the user, the display device 1005 can be omitted. Although not illustrated in FIG. 6, depending on the content of a process according to an exemplary embodiment, the computer 1000 may include an input device. For example, empirical risk estimation system 100 may include an input device for inputting an instruction to move to a link, such as clicking a portion where a link is set.

In addition, some or all of the component elements of each device are implemented by a general-purpose or dedicated circuitry, a processor or the like, or a combination thereof. These may be constituted by a single chip or may be constituted by a plurality of chips connected via a bus. In addition, some or all of the component elements of each device may be achieved by a combination of the above circuitry or the like and a program.

When some or all of the component elements of each device is achieved by a plurality of information processing devices, circuitries, or the like, the plurality of information processing devices, circuitries, or the like may be arranged concentratedly or distributedly. For example, the information processing device, circuitry, or the like may be achieved in the form in which a client and server system, a cloud computing system, and the like are each connected via a communication network.

### REFERENCE SIGNS LIST

10 density estimation unit

20 integral estimation unit

30 storage unit

100 empirical risk estimation system

What is claimed is:

1. An empirical risk estimation system comprising a hardware processor configured to execute a software code to:
estimate a conditional probability density of a random variable, denoting a real value that is a result of a smooth function map of given unobserved covariates, by training a regression model with a response corresponding to the random variable, and regressors corresponding to an observed covariates; and
estimate a one-dimensional integral of a product of a sigmoidal function with an input random variable and a conditional probability density function of the random variable.

2. The empirical risk estimation system according to claim 1, wherein the hardware processor is configured to execute a software code to:
estimate the conditional probability density of the random variable by a normal distribution; and
estimate the one-dimensional integral by using a piece-wise linear approximation of the sigmoid function.

3. An empirical risk estimation method comprising:
estimating a conditional probability density of a random variable, denoting a real value that is a result of a smooth function map of given unobserved covariates, by training a regression model with a response corresponding to the random variable, and regressors corresponding to an observed covariates; and
estimating a one-dimensional integral of a product of a sigmoidal function with an input random variable and a conditional probability density function of the random variable.

4. The empirical risk estimation method according to claim 3,
estimating the conditional probability density of the random variable by a normal distribution, and
estimating the one-dimensional integral by using a piece-wise linear approximation of the sigmoid function.

5. A non-transitory computer readable information recording medium storing an empirical risk estimation program, when executed by a processor, that performs a method for:
estimating a conditional probability density of a random variable, denoting a real value that is a result of a smooth function map of given unobserved covariates, by training a regression model with a response corresponding to the random variable, and regressors corresponding to an observed covariates; and
estimating a one-dimensional integral of a product of a sigmoidal function with an input random variable and a conditional probability density function of the random variable.

6. The non-transitory computer readable information recording medium according to claim 5, wherein
the conditional probability density of the random variable is estimated by a normal distribution, and
the one-dimensional integral is estimated by using a piece-wise linear approximation of the sigmoid function.
